(19) United States
(12) Patent Application Publication
(10) Pub. No.: US 2024/0051835 A1
(43) Pub. Date: Feb. 15, 2024

(54) AMORPHOUS NON-POROUS SILICAS

(71) Applicant: Evonik Operations GmbH, Essen, DE (US)

(72) Inventors: Nathan Motl, Bel Air, MD (US); Terry W, NASSIVERA, Gambrills, MD (US); Fitzgerald A. Sinclair, Smyma, DE (US); Scott Pentz, Havre de Grace, MD (US); Michael S. Darsillo, Landenberg, PA (US); Eric G. Lundquist, Essex, MD (US)

(73) Assignee: Evonik Operations GmbH, Essen, DE (US)

(21) Appl. No.: 18/264,963

(22) PCT Filed: Feb. 3, 2022
(86) PCT No.: PCT/EP2022/052516

§ 371 Date: Aug. 10, 2023

Related U.S. Application Data
(60) Provisional application No. 63/148,172, filed on Feb. 11, 2021.

(51) Int. Cl. C01B 33/187; A61K 8/25; A61Q 11/00

(52) U.S. Cl. CPC C01B 33/187; A61K 8/25; A61Q 11/00; C01P 2006/16; C01P 2004/32; C01P 2006/12; C01P 2004/61; C01P 2006/11; C01P 2006/14; C01P 2004/03; C01P 2004/04

(57) ABSTRACT

Spherical amorphous, non-porous silicas are useful for oral compositions. A method of making the spherical amorphous, non-porous silicas is also provided.

### TECHNICAL FIELD

The present disclosure relates generally to amorphous, non-porous silicas and methods of making and using the same in oral compositions.

### BACKGROUND

Silica exists in many different forms including both naturally occurring mineral and synthetic forms. Within the synthetic amorphous silica family, there exists silica gel, fumed silica, precipitated silica, fused silica and silica fume. These species of silica are characterized by their method of manufacture that yield specific final properties and structure. Silica gels, fumed silica and precipitated silica are generally porous materials made up of aggregates of primary particles. These silica products are generally formed by converting liquid silicate solutions or silanes into solid silica particles. Fused and silica fume tend to be solid, non-porous silica materials generally prepared via a melting process wherein these products go from a liquid state to a solid silica. Precipitated silica, silica gel and fumed silica have been used in cosmetic applications and oral care compositions such as toothpaste. Other silicas including fused silica, silica gels and precipitated silica that have been heated or calcined to either fuse the silica and/or reduce hydroxyl groups also have utility in oral care applications.

For example, U.S. Pat. No. 8,734,764 discloses oral compositions comprising fused silica as a dental abrasive. The fused silica disclosed therein can be produced by fusing silicon dioxide powder by flames of high temperatures (for example 2,000° C. to 3,000° C.) or by oxidizing and dissolving silicon powder or cyclic siloxane by high temperature flames. According to U.S. Pat. No. 8,734,764, since the silica is fused once and solidified, the silica particles have a high density and an increased hardness. The fused silica particles are disclosed as preferably having a high degree of sphericity i.e. their shape is close to be a complete sphere. BET specific surface area of the fused silica is disclosed as 15 m²/g or less, preferably 10 or less. Oil absorption is disclosed as usually 20 mL/100 g or less, preferably 10 mL/100 g. According to U.S. Pat. No. 8,734,764, oral composition comprising fused silica disclosed therein and a dental abrasive has a high stain removal ability relative to its abrasive ability, thereby making it possible to efficiently remove stains without damaging the teeth more than necessary.

WO 2014/071284 discloses precipitated silicas that have been subjected to rapid heat treatment (e.g., above 800° C.) to reduce surface hydroxyl on the silica while maintaining the inner precipiated silica structure, resulting in improved stability with metal ions such as stannous ions in an oral care composition.

WO 2019/068596 discloses spherical, anhydrous, amorphous silica gel particles having a pore volume of less than 0.1 ml/g useful in a dentifrice composition. The silica gel particles therein are non-fused, but rather are obtained by precipitation followed by calcination (i.e., heating to a high temperature but below melting or fusing point) to remove water to produce a dense (low porosity or non-porous) particulate silica material.

EP 0 216 278 discloses non-porous, spherical silica partilces with particle diameters between 0.05 and 10 microns and are highly monodisperse, made from hydrolytic polycondensation of tetraalkoxysilanes in aqueous alcoholic-ammoniakalisches medium.

U.S. Pat. Nos. 3,939,262 and 4,007,260 both describe dentifrice compositions containing finely divided synthetic amorphous silica polishing agents. Such amorphous silicas are made from particulate hydrous alkali metal silicates, particularly containing 10 to 25%, preferably 15 to 20% by weight of water. These silicates are prepared by dehydrating alkali metal silicate solutions by any known drying method such as spray-drying, drum-drying, and high pressure extrusion to yield substantially hollow shaped spheres or beads. The synthetic amorphous silicas are then prepared by adding the particulate hydrous silicates to an aqueous acid solution to yield silicas with a surface area (BET) of about 100 to about 200 m²/gm and upon milling, a particle size between 3 and 20 microns.

U.S. Pat. No. 4,312,845 discloses synthetic amorphous silica produced by hydrothermal treatment of an aqueous dispersion of silica and sodium hydroxide to form a partly polymerized sodium silicate that can be spray dried to form hollow spheres of polysilicate, and then treated with 5 to 15% sulfuric acid to form amorphous silica with a BET between 40 m²/g and 420 m²/g and an oil adsorption greater than 60 cc/100 g. For comparative studies, U.S. Pat. No. 4,312,845 repeated the process described in U.S. Pat. No. 3,939,262 to produce a sample having an oil adsorption of 33 cc/100 g.

JP5762120 discloses methods for producing silica-based particles having substantially no cavity in the interior portion of the particle, wherein the interior portion is porous or nonporous (not porous), or silica-based particles having an outer shell and having a cavity inside the outer shell, wherein the outer shell is porous or nonporous. The method comprises (a) a step of preparing silica-based particle precursor particles by spray-drying an alkali silicate aqueous solution in a hot air flow; (b) a step of immersing the silica-based particle precursor particles in an acid aqueous solution to remove the alkali; and (c) a step of drying and heat-treating the resulting particles. This references further teaches that “(C) the step of drying, heat treatment temperature in the range of 90-1200° C., the obtained silica-based particles in the outer shell of non-porous silica layer is preferable. (C) the step of drying, heat treatment is performed under reduced pressure, the obtained silica-based particles of the shell layer is preferably a negative pressure. When the average particle diameter is in the range of 0.1-200 μm, the outer shell has a hollow interior of the silica layer, the porosity of the cavity in the range of 20-95% by weight, nonporous silica layer and the outer shell, hollow inside and the negative pressure. The negative pressure inside the cavity is preferably less than or equal to 133hPa.”. JP5762120 also dislcoses the use of such silica particles in cosmetic and thermal insulation applications.

WO 2019/241323 discloses a dentrifice containing spherical precipitated silica particles that are created using a continuous loop reactor process. Specifically, the reactor loop contains a reaction slurry that is circulated numerous times before being discharged. Initially the reactor loop is filed wth a slurry that contains silica, sodium silicate, sodium sulfate, and water. Sulfuric acid is then added and the slurry is discharged as the volume increases. The resulting silica particles have a d50 median particle size from 4 to 25 μm, a BET Surface area of less than 10 m²/g and a total mecury intrusion pore volume from 0.2 to 1.5 mL/g.

The high-temperature process for preparing fused or calcined silicas are energy intensive and expensive and polycondensation of tetraalkoxysilanes require expensive materials such that the process may become cost prohibitive for many applications. Other processes produce porous and non-porous or hollow silica spheres for use in cosmetic and/or thermal insulation applications. In addition to the cost-prohibitive nature of manufacturing these materials, it has also been found that the higher degree of condensation of these particles leads to a higher abrasivity of hard surfaces such as metals and even tooth enamel. There remains a need in the art for a versatile, low-temperature process for producing amorphous non-porous silica, e.g., with good PCR, RDA and REA as well as compatible with fluoride and metal (e.g., stannous) ions.

### SUMMARY

The current inventors have discovered an efficient process for preparing amorphous non-porous silicas from acid neutralized spray-dried silica that provide benefits in dentrifice formulations. In contrast to non-porous silica particles which lack porosity or lack internal pore structure determined by several methods including but not limited to Electron microscopy including Scanning Electron Microscopy (SEM) and Transmission Electron Microscopy (TEM), porous silica particles have pores, or in the case of precipitated silica, primary particles that are agglomerated to form these channels that can be imaged and observed via these methods. The porosity of porous silica particles can also be determined by physical methods such as Nitrogen Porosimetry and Mercury Porosimetry in which surface area and pore volume can be obtained. Non-porous silica particles generally have low nitrogen adsorption pore volume, low Mercury Intrusion pore volume and/or low BET surface areas. Other surface area measurement methods include CTAB and Acorn which utilize probe molecules cetyl trimethylammounium bromide (CTAB) and water (Acorn) to measure surface area. Other physical methods such as absorbtion of oil or water can also be used to determine porosity. A non porous material will have low absorption properties while a porous material will have high absorption properties. In these absorption methods, some degree of absorption will occur even in non porous materials as space between particles is also included. Additionally, porosity impacts the surface of the material imparting a rough surface like a sponge. Non porous materials have no structure and thus a smooth surface like a glass. The inventors have found that a smooth surface provides benefit in the RDA of a material at a given density. Therefore, in the first aspect, the invention provides the following:
1.1. an oral composition comprising amorphous, non-porous silica particles that are not produced via polycondensation of a tetraalkoxysilane or a precipitation process or heat treated to over 200° C., wherein the RDA is less than 200, in another embodiment, less than 180, and a total mercury intrusion (HgI) pore volume of such silica particles is less than or equal to 0.8 cc/g or an intraparticle HgI pore volume of such silica particles at a pore diameter of less than 0.11 μm is less than or equal to 0.1 cc/g;
1.2. the oral composition of formula 1.1, wherein the silica particles are spherical;
1.3. the oral composition of formula 1.1 or 1.2, wherein the BET surface area of such silica particles is less than 25 m²/g, in one embodiment, less than 10 m²/g, in another embodiment, less than 3 m²/g; in another embodiment, less than 1.5 m²/g;
1.4. the oral composition of formula 1.1, 1.2 or 1.3, wherein the median particle size of such silica particles is between 1 and 25 μm, in one embodiment, between 1 and 15 μm, in another embodiment, between 3 and 10 μm;
1.5. the oral composition of formula 1.1, 1.2, 1.3 or 1.4, wherein the oil absorption capacity of such silica particles is below or equal to 40 cc/100 g, in another embodiment, between 15 and 30 cc/100 g;
1.6. the oral composition of any of formulae 1.1-1.5, wherein the pack density of such silica particles is greater than 60 lb/ft³, in one embodiment, greater than or equal to 62 lb/ft³, in another embodiment, between 62 and 70 lb/ft³;
1.7. the oral composition of any formulae 1.1-1.6, wherein the pour density of such silica particles is less than 50 lb/ft³, in one embodiment, less than 45 lb/ft³, in another embodiment, between 40 and 45 lb/ft³;
1.8. the oral composition silica of any formulae 1.1-1.7, wherein the loss on ignition (LOI) of such silica particles is between 3 and 10 wt. %, in one embodiment, between 3 and 8 wt. %, in another embodiment, between 3 and 7 wt. %, in still another embodiment, between 6 and 10 wt. %, in another embodiment, between 6 and 8 wt. %, in yet another embodiment, between 5 and 8 wt. %;
1.9. the oral composition of any formulae 1.1-1.8, wherein the Acorn surface area of such silica particles is less than 10 m²/g, in another embodiment, less than 5 m²/g, in another embodiment less than 2 m²/g, in still another embodiment, between 0.1 and 3 m²/g, in yet another embodiment, between 0.5 and 2 m²/g;
1.10. the oral composition of any formulae 1.1-1.9, wherein the CTAB surface area of such silica particles is less than 15 m²/g, in one embodiment, less than 12 m²/g, in another embodiment, less than 10 m²/g, in still another embodiment, between 1 and 15, in yet another embodiment, between 1 and 12 m²/g, in still another embodiment, between 1 and 10 m²/g, in yet another embodiment, between 1 and 6 m²/g;
1.11. the oral composition of any formulae 1.1-1.10, wherein the total mercury intrusion pore volume of such silica particles is less than 0.7 cc/g, in one embodiment, less than or equal to 0.6 cc/g, in still another embodiment, less than or equal to 0.4 cc/g, in another embodiment, less than or equal to 0.3 cc/g, in still another embodiment, between 0.1 and 0.5 cc/g, in yet another embodiment, between 0.3 and 0.4 cc/g;
1.12. the oral composition of any formulae 1.1-1.11, wherein the intraparticle mercury intrusion volume of such silica particles of pores less than 0.11 microns in diameter of such silica particles is less than 0.10 cc/g in one embodiment, less than or equal to 0.07 cc/g in another embodient, in still another embodiment less than or equal to 0.05 cc/g;
1.13. the oral composition of any formulae 1.1-1.12, wherein the 5% pH of such silica particles is between 4 and 11, in one embodiment, between 4 and 10, in still another embodiment, between 6 and 10, in one embodiment between 5 and 8, in yet another embodiment, greater than or equal to 7, in still another embodiment, greater than or equal to 8;
1.14. the oral composition of any formulae 1.1-1.13, wherein the moisture corrected water AbC value of such silica particles is less than 45 cc/100 g, in another embodiment, less than 40 cc/100 g, in still another embodiment, between 25 cc/100 g and 42 cc/100 g, in yet another embodiment, between 30 cc/100 g and 40 cc/100 g;
1.15. the oral composition of any formulae 1.1-1.14, wherein the Einlehner abrasion value of such silica particles is less than or equal to 10 mg, in another embodiment, less than or equal to 7 mg, in still another embodiment, less than 6 mg, in yet another embodiment, between 1 and 9 mg loss per 100,000 revolutions;
1.16. the oral composition of any formulae 1.1-1.15, wherein the BJH nitrogen adsorption pore volume of such silica particles is less than 0.002 cc/g, in one embodiment, less than 0.001 cc/g;
1.17. the oral composition of any formulae 1.1, wherein such silica particles are non-spherical (for example plate-like or irregular or angular shape) and have one or more of the following properties:
   (a) BET surface area is less than 25 m²/g, in one embodiment, less than 10, in another embodiment, less than 3 m²/g, in still another embodiment, less than 1.5 m²/g;
   (b) a median particle is between 1 and 25 μm, in one embodiment, between 1 and 15 μm, in another embodiment, between 3 and 10 μm;
   (c) the oil absorption capacity is less than 50 cc/100 g, in another embodiment, between 30 and 50 cc/100 g, in still another embodiment, between 30 and 45 cc/100 g;
   (d) the pack density is greater than 35 lb/ft³, in one embodiment, between 35 and 60 lb/ft³;
   (e) pour density of such silica particles is less than 40 lb/ft³, in one embodiment, between 20 and 30 lb/ft³;
   (f) the loss on ignition (LOI) is between 3 and 10 wt. %, in one embodiment, between 3 and 8 wt. %, in another embodiment, between 3 and 7 wt. %, in still another, between 6 and 10 wt. %, in another embodiment, between 6 and 8 wt. %, in yet another embodiment, between 5 and 8 wt. %;
   (g) the Acorn surface area is less than 10 m²/g, in another embodiment, less than 5 m²/g, in another embodiment less than 2 m²/g, in still another embodiment, between 1 and 10 m²/g;
   (h) the CTAB surface area of such silica particles is less than 15 m²/g, in one embodiment, less than 5 m²/g, in another embodiment, less than or equal to 2 m²/g;
   (i) the total mercury intrusion volume of such silica particles is less than or equal to 0.8 cc/g, in one embodiment, between 0.5 and 0.8 cc/g;
   (j) the intraparticle mercury intrusion pore volume of such silica particles of pores less than 0.11 microns in diameter of such silica particles is less than 0.10 cc/g in one embodient, less than or equal to 0.07 cc/g in another embodient, in still another embodient less than or equal to 0.05 cc/g;
   (k) the 5% pH is between 4 and 11, in one embodiment, between 6 and 10, in still another embodiment, between 4 and 10, in one embodiment between 5 and 8, in yet another embodiment, greater than or equal to 7, in still another embodiment, greater than or equal to 8;
   (l) the moisture corrected water AbC value is less than 90 cc/100 g, in another embodiment, less than 80 cc/100 g, in still another embodiment, less than 65 cc/100 g;
   (m) Einlehner abrasion value is less than or equal to 30 mg, in another embodiment, less than or equal to 25 mg, in still another embodiment, between 10 and 25 mg loss per 100,000 revolutions;
   (n) BJH nitrogen adsorption pore volume of such silica particles is less than 0.02 cc/g, in one embodiment, less than 0.01 cc/g; and/or
   (o) any combination of (a)-(n) thereof;
1.18. the oral composition of any formulae 1.1-1.17 wherein the Scanning Electron Microscopy (SEM) and/or Transmission Electron Microscopy (TEM) of such silica particles shows no evidence of an internal structure or porosity;
1.19. the oral composition of any of the foregoing formulae, wherein such silica particles are made from the process of any of formulae 2.1-2.16;
1.20. the oral composition of any of the foregoing formulae, wherein the composition is an oral care composition comprising one or more orally acceptable carrier, in one embodiment, the oral care composition is a dentifrice composition;
1.19. the oral composition of any of the foregoing formulae, wherein the loading level of silica is between 1 and 20 wt. %, in one embodiment, between 5 and 20 wt. %, in another embodiment, between 10 and 20 wt. %, based on the total weight of the composition;
1.20. the oral composition of any of formulae 1.1-1.19, wherein the Pellicle Cleaning Ratio (PCR) is greater than 70, in one embodiment, greater than 85, in another embodiment between 85 and 110;
1.21. the oral composition of any of formulae 1.1-1.20, wherein the relative dentine abrasive (RDA) value is less than or equal to 180, in another embodiment, less than 150, in still another embodiment, between 50 and 150;
1.22. the care composition of any of formulae 1.1-1.21, wherein said composition has a fluoride compatibiilty of greater than or equal to 50%, in one embodiment, greater than 60%, in another embodiment, greater than 70%, in still another embodiment, greater than 80%, in yet embodiment, greater than or equal to 90%, in still another embodiment, greater than or equal to 95%;
1.23. the oral composition of formula 1.1-1.22, wherein said composition has a stannous ion compatibility of greater than or equal to 50%, in one embodiment, greater than or equal to 60%, in another embodiment, greater than or equal to 70%, in still another embodiment, greater than or equal to 80%, in still another embodiment, greater than or equal to 90%, in yet another embodiment, greater than or equal to 95%:
1.24. the oral composition of any of the foregoing formulae, wherein the Relative Enamel abrasion is less than or equal to 15 in one embodiment, in another embodiment less than 10, in yet another embodiment less than 5.

In the second aspect, the invention provides the following:
2.1. a process for preparing amorphous non-porous silica particles comprising the steps of (i) spray drying an alkali metal silicate solution to form silicate powder; (ii) neutralizing the silicate powder of step (i) with an acid;
2.2. the process of formula 2.1, wherein the spray drying step (i) comprises atomization of the sodium silicate using a spray nozzle or pneumatic nozzle (e.g., using pressure energy);
2.3. the process of formula 2.1 or 2.2, wherein the nozzle diameter is between 1 and 2 mm;
2.4. the process of formula 2.1 or 2.3, wherein the nozzle pressure is between 1 and 5 bar, in one embodiment between 1 and 3 bar;
2.5. the process of formula 2.1, wherein the spray drying step (i) comprises atomization using a rotary atomizer (e.g., centrifugal energy);
2.6. the process of any of formulae 2.1-2.5, wherein the neutralization temperature of the alkali metal silicate is less than or equal to 30° C., in one embodiment, at room temperature, in another embodiment, less than 25° C., in still another embodiment, less than 10° C., in yet another embodiment, less than 0° C.;
2.7. the process of any of formulae 2.1-2.6, wherein step (i) and (ii) are carried out at atmospheric pressure;
2.8. the process of any of formulae 2.1-2.7, wherein the molar ratio of silica: alkali metal oxide is between 2.5 and 3.5, in one embodiment, between 2.5 and 3.3, in another embodiment between 2.65 and 3.3;
2.9. the process of any of formulae 2.1-2.8, wherein the alkali metal is selected form the group consisting of sodium, potassium, lithium or any mixture thereof, in one embodiment, the alkali metal silicate is sodium silicate;
2.10. the process of any of formulae 2.1-2.9, wherein the silicate concentration is between 10 and 15 wt. %, in one embodiment, at 15 wt. %;
2.11. the process of any of formulae 2.1-2.10, wherein the neutralization step (ii) comprises adding the resulting silicate powder of step (i) to the acid;
2.12. the process of any of formulae 2.1-2.11, wherein the acid is a mineral acid, in one embodiment, selected from the group consisting of sulfuric acid, hydrochloric acid, nitric acid, phosphoric acid, carbonic acid or a combination thereof, in another embodiment, the acid is a sulfuric acid; in still another embodient, the acid is carbonic acid.
2.13. the process of any of formulae 2.1-2.12, wherein the concentration of acid is less than 10 wt. %, in one embodiment, less than 6 wt. %, in another embodiment at 5 wt. %;
2.14. the process of any of formulae 2.1-2.13, wherein milling is required;
2.15. the process of any of formulae 2.1-2.13, wherein no milling is required;
2.16. the process of any of formulae 2.1-2.15, wherein the acid is adjusted to provide a final pH of the dried silica of greater than or equal to 7 in one embodiment, in another embodiment, greater than or equal to 8; in still another embodiment, the silica is post-treated wherein 5 wt. % of the silica particles is adjusted to a pH of greater 7, in one embodiment, greater than 8, in another embodiment, using e.g., a base such as sodium bicarbonate.

### BRIEF DESCRIPTION OF THE DRAWING

FIG. 1 shows the scanning electron microscopy (SEM) images of examples 1A-1I at 2K×Magnification and scale bars at 10 μm.

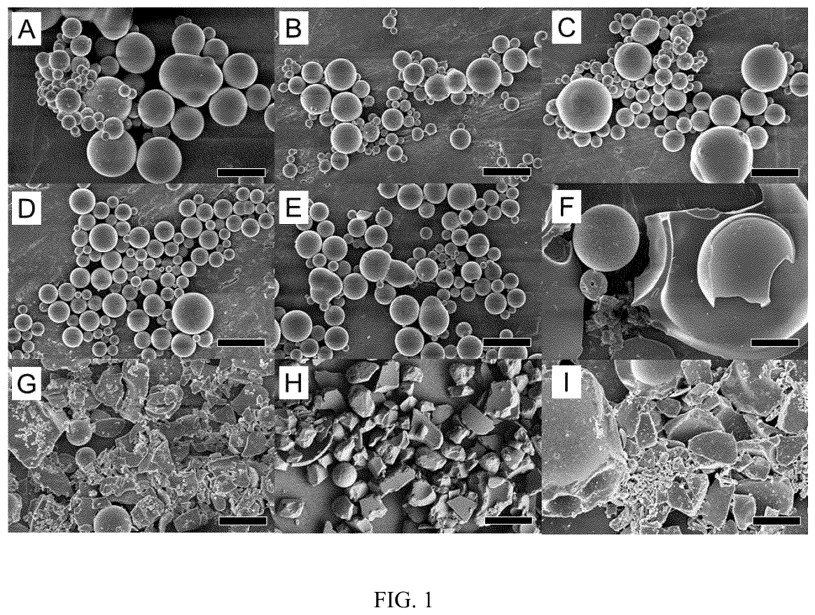

FIG. 2 shows SEM Images of comparative examples 2 and 4 alongside inventive examples 1B and 1H.

### DETAILED DESCRIPTION

The current invention provides amorphous non-porous silica particles and methods of making the same and their use in dentrifice compositions. Amorphous, non-porous silica silicas of the invention are not produced by a polycondensation of a tetraalkoxysilane and are not produced by a precipitation process and are not produced by a heat treatment or calcination process.

The inventors have discovered that the amorphous non-porous silicas can be made by controlling the alkali metal silicate solution, the spray drying parameters, and the neutralization step. It was discovered that when the alkali metal silicate is spray dried via pressure atomization such as using a spray nozzle, smaller dense spheres can be made while using rotary atomizer will yield larger hollow spheres which are then milled to non-spherical silicas. Spherical means the silicas are rounded to well-rounded in nature (non-angular or non-plate-like), in a particular embodiment, at least 50% of the particles are rounded to well rounded. Non-spherical means angular or plate-like, in particular embodiment, at least 50% of the particles are angular or plate-like. Therefore, the invention provides processes for preparing amorphous non-porous silicas comprising the steps of (i) spray drying a an alkali metal silicate solution to form silicate powder; (ii) neutralizing the silicate powder of step (i) with an acid. Preferably, the spray drying step (i) comprises atomization of the sodium silicate using a spray nozzle or pneumatic nozzle (e.g., using pressure energy), e.g., with a diameter between 1 and 2 mm. Preferably, the nozzle pressure is between 1 and 6 bar, in one embodiment between 1 and 3 bar.

It was also discovered that the temperature or rate of the neutralization can also affect the morphology of the silicas. The silicate precursor can be considered as insoluble under ambient conditions, but additional heat will partially solubilize the silicate. Soluble sodium siicate will produce precipitated silica decorating the monolithic spheres, resulting in an increase of BET surface area. This effect can be mitigated by either slowing the silicate addition to the acid solution, using dilute concentrations, or performing the addition under chilled conditions. Therefore, the spray-dried alkali metal silicate is preferably neutralized by addition to a dilute acid solution. In one embodiment, the concentration is less than 10 wt. %, in another embodiment, less than 6 wt. %, in still another embodiment at 5 wt. %. By adjusting the concentration of the acid solution, it is possible to control the pH of the resulting silica without impacting the morphology. Morphology is preserved during neutralization; the silicate will convert to silica without a change in the particle size distribution. The process of the current invention therefore does not require heating the silicate nor the silica at elevated temperature as taught in the prior art (e.g., fusing or calcining at high temperature), thereby producing silicas without modifying its surface chemistry and other properties.

The acid useful for the processes of the invention is a mineral acid including without limitation, selected from the group consisting of sulfuric acid, hydrochloric acid, nitric acid, phosphoric acid, carbonic acid or a combination thereof, in another embodiment, the acid is sulfuric acid, in still another embodiment, the acid is carbonic acid.

In addition to the concentration of the acid, the neutralization temperature of the alkali metal silicate is preferably at ambient temperature, therefore, in one embodiment, less than 30° C., in another embodiment, less than 25° C., in still another embodiment, less than 10° C., in yet another embodiment, less than 0° C.

The processes of the invention can utilize any silicate source including without limitation, alkali metal silicate wherein in one embodiment, the alkali metal is selected form the group consisting of sodium, potassium, lithium or any mixture thereof. In another embodiment, the alkali metal silicate is sodium silicate, in still another embodiment, the sodium silicate is rice hull silicate derived from the extraction of silca using caustic from either rice hulls or rice hull ash.

The molar ratio of the silica to alkali metal oxide for the alkali metal silicate is preferably 2.5-3.5, in a particular embodiment, selected from 2.5, 3, 3.3 and 3.5, in still another embodiment, between 2.5 and 3.3, in another embodiment, between 2.6 and 3.3. Preferably, the silicate concentration is between 10 and 30 wt. %, in one embodiment, about 15 wt. %.

To avoid changing the morphology of the silica particles, the spray-dried silicate is preferably added under condition to mitigate unwanted rise in temperatures due to the neutralization reaction.

The process of the invention described herein, particularly using the pneumatic nozzle, yields amorphous non-porous silica particles with no internal structure and low porosity or no porosity and a smooth surface.

The amorphous non-porous silica particles of the invention generally have a BET surface area is less than 25 m²/g, in one embodiment, less than 10 m²/g, in another embodiment, less than 3 m²/g; in another embodiment, less than 1.5 m²/g. Non-spherical non-porous silica particles of the invention generally have a nitrogen adsorption pore volume of less than 0.03 cc/g, in one embodiment, less than 0.02 cc/g, in another embodiment, less than 0.01 cc/g, in still another embodiment, less than 0.005 cc/g, while spherical non-porous silica particles of the invention generally have a nitrogen adsorption pore volume of less than 0.002 cc/g, in still another embodiment, less than 0.001 cc/g. BET surface areas and the nitrogen adsorption pore volume of the silica particles may be determined with any known methods in the art such as a Micromeritics TriStar 3020 instrument using the BET nitrogen adsorption method of Brunaur et al., J. Am. Chem. Soc., 60, 309 (1938) known in the field of silica and silicate materials and the nitrogen adsorption total pore volume method known in the field of silica and silicate materials (Pure Appl. Chem. 2015; 87(9-10): 1051-1069), the contents of each of which are hereby incorporated by reference in their entirety.

The silicas of the invention also generally have a median particle size (d50) between 1 and 25 μm, in one embodiment, between 1 and 15 μm, in another embodiment, between 3 and 10 μm. The median particle size (d50) refers to the particle size for which 50% of the sample has a smaller size and 50% of the sample has a larger size. Particle size can be determined by laser diffraction method using, e.g., a Horiba LA 300 instrument.

The spherical silicas of the invention generally have an oil absorption capacity below 40 cc/100 g, in another embodiment, between 15 and 30 cc/100 g while the non-spherical silicas of the invention have an oil absorption capacity of less than 50 cc/100 g, in another embodiment, between 30 and 50 cc/100 g, in still another embodiment, between 30 and 45 cc/100 g. Oil absorption values may be determined in accordance with the rub-out method described in ASTM D281 using linseed oil (cc oil absorbed per 100 g of the particles). Generally, a higher oil absorption level indicates a particle with a higher level of large pore porosity, also described as higher structure.

The spherical silicas of the invention generally have a pack density of greater than 60 lb/ft³, in one embodiment, greater than or equal to 62 lb/ft³, in another embodiment, between 62 and 70 lb/ft³. The spherical silicas of the invention also generally have a pour density of less than 50 lb/ft³, in one embodiment, less than 45 lb/ft³, in another embodiment, between 40 and 45 lb/ft³. The non-spherical silicas of the invention have a pack density of greater than 35 lb/ft³, in one embodiment, between 35 and 60 lb/ft³ and pour density of less than 40 lb/ft³, in one embodiment, between 20 and 30 lb/ft³. Pack density and pour density may be measured by placing a weighted sample into a 250 mL graduated cylinder with a flat rubber bottom. The initial volume is recorded and used to calculate the pour density by dividing it into the weight of sample used. The cylinder is then placed onto a tap density machine where it is rotated on a cam at a specific RPM. The cam is designed to raise and drop the cylinder a distance of 5.715 cm once per second, until the sample volume is constant, typically for 15 min. This final volume is recorded and used to calculate the packed density by dividing it into the weight of sample used.

The silicas of the invention generally have a loss on ignition (LOI) of between 3 and 10 wt. %, in one embodiment, between 3 and 8 wt. %, in another embodiment, between 3 and 7 wt. %, in still another embodiment, between 6 and 10 wt. %, in another embodiment, between 6 and 8 wt. %, in yet another embodiment, between 5 and 8 wt. %. LOI may be measured by first weighing approximately 2 g sample (to the nearest 0.0001 g) and transfering the sample into a preignited and cooled crucible. The sample must be dried 2 hours at 105° C., cooled to room temperature in a desiccator before weighing. The sample is then heated for 2 hrs in a muffle furnace at a temperature of 1000C. After heating, the sample is removed and cooled in a dessicator. The sample weight is then measured. The % LOI is then calculated by dividing the weight loss by the sample weight before heating.

The silicas of the invention generally have an Acorn surface area of less than 10 m²/g, in another embodiment, less than 5 m²/g, in another embodiment less than 2 m²/g, in still another embodiment, between 1 and 10 m²/g, in yet another embodiment, between 0.1 and 3 m²/g, in yet another embodiment, between 0.5 and 2 m²/g. Acorn surface area may be measured by Xigo Acorn Area Surface area Analyzer.

The silicas of the invention generally have a CTAB surface area of less than 15 m²/g, in one embodiment, less than 12 m²/g, in another embodiment, less than 10 m²/g, in still another embodiment, between 1 and 15, in yet another embodiment, between 1 and 12 m²/g, in still another embodimnet, between 1 and 10 m²/g, in yet another embodiment, between 1 and 6 m²/g. CTAB surface areas of the silicas of the invention may be measured by absorption of CTAB (cetyltrimethylammonium bromide) on the silica surface, wherein the excess CTAB are separated by centrifugation and the quantity determined by titration with sodium lauryl sulfate using a surfactant electrode, for example, using the American Standard Test Method D6845-20.

The spherical silicas of the invention generally have total mercury intrusion volume of less than 0.7 cc/g, in one embodiment, less than 0.6 cc/g, in another embodiment, less than or equal to 0.4 cc/g, in another embodiment, less than or equal to 0.3 cc/g, in still another embodiment, between 0.1 and 0.5 cc/g, in yet another embodimnet, between 0.3 and 0.4 cc/g. The non-spherical silicas of the invention generally have a total mercury intrusion volume is less than or equal to 0.8 cc/g, in one embodiment, between 0.5 and 0.8 cc/g. Both the spherical and non spherical silicas of the present invention have an intraparticle mercury intrusion pore volume of pores less than 0.11 microns in diameter of less than 0.10 cc/g in one embodient, less than or equal to 0.07 cc/g in another embodient, in still another embodient less than or equal to 0.05 cc/g.

Mercury intruded volume or total pore volume (Hg) is measured by mercury porosimetry using a Micromeritics AutoPore IV 9520 (or Micromeritics AutoPore V 9620) apparatus. The pore diameters can be calculated by the Washburn equation employing a contact angle Theta (Θ) equal to 1300 and a surface tension gamma equal to 484 dynes/cm. Mercury is forced into the voids of the particles as a function of pressure and the volume of the mercury intruded per gram of sample is calculated at each pressure setting. Total pore volume expressed herein represents the cumulative volume of mercury intruded at pressures from vacuum to 60,000 psi. Increments in volume (cm3/g) at each pressure setting are plotted against the pore radius or diameter corresponding to the pressure setting increments. The peak in the intruded volume versus pore radius or diameter curve corresponds to the mode in the pore size distribution and identifies the most common pore size in the sample. Specifically, sample size is adjusted to achieve a stem volume of 25-90% in a powder penetrometer with a 5 mL bulb and a stem volume of about 1.1 mL. Samples are evacuated to a pressure of 50 μm of Hg and held for 5 minutes. Mercury fills the pores from 4.0 to 60,000 psi with a 10 second equilibrium time at each data collection point. The total pore volume as described above captures the volumes from intraparticle porosity resulting from the pore structure within the individual particles, as well as, the interparticle porosity formed from the interstitial spacing of the packed particles under pressure. To better separate and measure the actual intraparticle porosity of the produced amorphous silicas, the pore volume of pores less than <0.11 μm can be used.

The silicas of the invention generally have 5% pH of between 4 and 11, in one embodiment, between 4 and 10, in still another embodiment, between 6 and 10, in one embodiment between 5 and 8, in yet another embodiment, greater than or equal to 7, in still another embodiment, greater than or equal to 8. 5% pH may be measured by determining the pH of an aqueous system containing 5 wt. % solids in deionized water using a pH meter.

The spherical silicas of the invention generally have a water corrected AbC value of less than 45 cc/100 g, in another embodiment, less than 45 cc/100 g, in another embodiment, less than 40 cc/100 g, in still another embodiment, between 25 cc/100 g and 42 cc/100 g, in yet another embodiment, between 30 cc/100 g and 40 cc/100 g. The non-spherical silicas of the invention generally have a water corrected AbC value of less than 90 cc/100 g, in another embodiment less than 80 cc/100 g, in still another embodiment, less than 65 cc/100 g. AbC values may be determined with an Absorptometer “C” torque rheometer from C.W. Brabender Instruments, Inc.

The spherical silicas of the invention generally have Einlehner abrasion value of less than or equal to 10 mg, in another embodiment, less than or equal to 7 mg, in still another embodment, less than 6 mg, in yet another embodiment, between 1 and 9 mg loss per 100,000 revolutions. The non-spherical silicas of the invention generally have Einlehner abrasion value of less than or equal to 30 mg, in another embodiment, less than or equal to 25 mg, in still another embodiment, between 10 and 25 mg loss per 100,000 revolutions. The Brass Einlehner Abrasion (BEA) test used to measure the hardness of the silica products of the invention is described in detail in U.S. Pat. No. 6,616,916 to Karpe et al., which is incorporated herein by reference for its teaching of the BE Abrasion test. Generally, the test involves an Einlehner AT-1000 Abrader used as follows: (1) a Fourdrinier brass wire screen that has been previously cleaned and dried is then weighed and exposed to the action of a 10% aqueous silica suspension for a fixed length of time, specifically 100 g of sample in 900 g of deionized water; (2) the amount of abrasion is then determined as milligrams brass lost from the Fourdrinier wire screen per 100,000 revolutions. The result, measured in units of mg loss, can be characterized as the 10% brass Einlehner (BE) abrasion value.

In another aspect, wherein the process of the invention utilizes a rotary atomizer, such process produces dense hollow shells which can be milled and which have one or more of the following properties:
(a) BET surface area is less than 25 m²/g, in one embodiment, less than 10, in another embodiment, less than 3 m²/g, in still another embodiment, less than 1.5 m²/g;
(b) a median particle is between 1 and 25 μm, in one embodiment, between 1 and 15 μm, in another embodiment, between 3 and 10 μm;
(c) the oil absorption capacity is less than 50 cc/100 g, in another embodiment, between 30 and 50 cc/100 g, in still another embodiment, between 30 and 45 cc/100 g;
(d) the pack density is greater than 35 lb/ft³, in one embodiment, between 35 and 60 lb/ft³;
(e) pour density of such silica particles is less than 40 lb/ft³, in one embodiment, between 20 and 30 lb/ft³;
(f) the loss on ignition (LOI) is between 3 and 10 wt. %, in one embodiment, between 3 and 8 wt. %, in another embodiment, between 3 and 8 wt. %, in another embodiment, between 3 and 7 wt. %, in still another, between 6 and 10 wt. %, in another embodiment, between 6 and 8 wt. %, in yet another embodiment, between 5 and 8 wt. %;
(g) the Acorn surface area is less than 10 m²/g, in another embodiment, less than 5 m²/g, in another embodiment less than 2 m²/g, in still another embodiment, between 1 and 10 m²/g;
(h) the CTAB surface area of such silica particles is less than 15 m²/g, in one embodiment, less than 5 m²/g, in another embodiment, less than or equal to 2 m²/g;
(i) the total mercury intrusion volume is less than or equal to 0.8 cc/g, in one embodiment, between 0.5 and 0.8 cc/g;
(j) the intraparticle mercury intrusion pore volume of pores less than 0.11 microns in diameter of such silica particles is less than 0.10 cc/g in one embodient, less than or equal to 0.07 cc/g in another embodient, in still another embodient less than or equal to 0.05 cc/g;
(k) the 5% pH is between 4 and 11, in one embodiment, between 4 and 10, in still another embodiment, between 6 and 10, in one embodiment between 5 and 8, in yet another embodiment, greater than or equal to 7, in still another embodiment, greater than or equal to 8;
(l) the moisture corrected water AbC value is less than 90 cc/100 g, in another embodiment, less than 80 cc/100 g, in still another embodiment, less than 65 cc/100 g;
(m) Einlehner abrasion value is less than or equal to 30 mg, in another embodiment, less than or equal to 25 mg, in still another embodiment, between 10 and 25 mg loss per 100,000 revolutions; and/or
(n) any combination of (a)-(m) thereof.

The silicas of the current invention can be used in oral care applications, such as in a dentifrice composition wherein such composition comprises one or more orally acceptable carrier.

In dentrifice applications, the inventors have surprisingly found that the non-porous amorphous silica particles give unexpectantly high cleaning and low abrasion for a silica with relatively high density. In dentrifice formulations, silica provides cleaning as measured by PCR (Pelicle Cleaning Ratio). Although high PCR is desired to provide high cleaning toothpaste, it must be tempered with low RDA (Relative Dentin Abrasion) and low REA (Relative Enamel Abrasion) to provide a suitable toothpaste that does not damage the tooth dentin or tooth enamel. For precipitated silicas, silica particles with higher pack density and lower mercury intrusion pore volume (lower porosity) give higher abrasion as disclosed in U.S. Pat. No. 10,328,002. The inventors have surprisingly discovered that the amporphous non-porous silicas of the present invention provide comparable high cleaning (PCR) and lower abrasion (RDA) than other silicas that have lower densities and higher porosities.

The dentifrice composition can contain any suitable amount of the silica particles, such as from about 0.5 to about 50 wt. %, from about 1 to about 50 wt. % in a particular aspect, from about 1 to about 35 wt. % in a particular aspect, from about 1 to about 20 wt. % in a particular aspect, and from about 10 to about 20 wt. % in a more particular aspect, of the amorphous, non-porous silica particles of the invention. These weight percentages are based on the total weight of the oral (e.g., dentifrice) composition.

The dentifrice compositions disclosed herein can be evaluated using a variety of measurements. The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR may be determined by any method known in the art, one of which is described in “In Vitro Removal of Stain With Dentifrice” G. K. Stookey, et al., J. Dental Res., 61, 1236-9, 1982, which is incorporated herein by reference for its teaching of PCR. In one embodiment, the oral care (e.g., dentifrice) composition of the invention have a Cleaning Ratio (PCR) is greater than 70, in one embodiment, greater than 85, in another embodiment between 85 and 110.

In another embodiment, the oral care (e.g., dentifrice) composition of the invention have a relative dentine abrasive (RDA) value of less than or equal to 180, in another embodiment, less than 150, in still another embodiment, between 50 and 150. RDA values of dentifrices containing the silica particles used in this invention may be determined according to the method set forth by Hefferen, Journal of Dental Res., July-August 1976, 55 (4), pp. 563-573, and described in Wason U.S. Pat. Nos. 4,340,583, 4,420,312 and 4,421,527, which are each incorporated herein by reference for their teaching of RDA measurements.

In another embodiment, the oral care (e.g., dentifrice) composition of the invention have a relative enamel abrasive (REA) value of less than or equal to 15 in one embodient, in another embodient less than 10, in yet another embodient less than 5. REA values of dentifrices containing the silica particles used in this invention may be determined according to the method known in the art, onw of which is set forth by Hefferen, Journal of Dental Res., July-August 1976, 55 (4), pp. 563-573, and described in Wason U.S. Pat. Nos. 4,340,583, 4,420,312 and 4,421,527, which are each incorporated herein by reference for their teaching of REA measurements.

In still another embodiment, the oral care (e.g., dentifrice) composition of the invention have a fluoride compatibiilty of greater than or equal to 80%, in one embodiment, greater than or equal to 90%, in another embodiment, greater than or equal to 95%; and/or a stannous ion compatibility of greater than or equal to 50%, in one embodiment, greater than or equal to 60%, in another embodiment, greater than or equal to 70%, in still another embodiment, greater than or equal to 80%, in still another embodiment, greater than or equal to 90%, in yet another embodiment, greater than or equal to 95%. Fluoride compatibility may be determined by methods known in the art.

The dentifrice composition can be in any suitable form, such as a liquid, powder, gel or paste. In addition to the silica particles, the dentifrice composition comprises one or more orally acceptable carrier suitable for use in an oral cavity, which may include other ingredients or additives, non-limiting examples of which can include a humectant, a solvent, a binder, a therapeutic agent, a chelating agent, a thickener other than the silica particles, a surfactant, an abrasive other than the silica particles of the invention, a sweetening agent, a colorant, a flavoring agent, a preservative, and the like, as well as any combination thereof.

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, and mixtures thereof. In some formulations, humectants are present in an amount from about 20 to about 50 wt. %, based on the weight of the dentifrice composition.

A solvent can be present in the dentifrice composition, at any suitable loading, and usually the solvent comprises water. When used, water is preferably deionized and free of impurities, can be present in the dentifrice at loadings from 5 to about 70 wt. %, and from about 5 to about 35 wt. % in another aspect, based on the weight of dentifrice composition.

Therapeutic agents also can be used in the compositions of this invention to provide for the prevention and treatment of dental caries, periodontal disease, and temperature sensitivity, for example. Suitable therapeutic agents can include, but are not limited to, fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate; tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quaternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; hydrogen peroxide, peroxides and the like. Therapeutic agents can be used in dentifrice formulations singly or in combination, and at any therapeutically safe and effective level or dosage.

Thickening agents are useful in the dentifrice compositions to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof. Typical levels of thickening agents or binders are up to about 15 wt. % of a toothpaste or dentifrice composition.

Useful silica thickeners for utilization within a toothpaste composition, for example, include, as a non-limiting example, an amorphous precipitated silica such as ZEODENT 165 silica. Other non-limiting silica thickeners include ZEODENT 153, 163 and/or 167 and ZEOFREE, 177, and/or 265 silica products, all available from Evonik Corporation.

Surfactants can be used in the dentifrice compositions of the invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the compositions of the present invention in an amount from about 0.1 to about 15 wt. %, from about 0.3 to about 5 wt. % in a particular aspect, and from about 0.3 to about 2.5 wt. % in a more particular aspect.

The disclosed silica particles of the invention can be utilized alone as the abrasive in the dentifrice composition, or as an additive or co-abrasive with other abrasive materials discussed herein or known in the art. Thus, any number of other conventional types of abrasive additives can be present within the dentifrice compositions of the invention. Other such abrasive particles include, for example, precipitated calcium carbonate (PCC), ground calcium carbonate (GCC), chalk, bentonite, dicalcium phosphate or its dihydrate forms, silica gel (by itself, and of any structure), precipitated silica, amorphous precipitated silica (by itself, and of any structure as well), perlite, titanium dioxide, dicalcium phosphate, calcium pyrophosphate, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. Such materials can be introduced into the dentifrice compositions to tailor the polishing characteristics of the target formulation.

Sweeteners can be added to the dentifrice composition (e.g., toothpaste) to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Flavoring agents also can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents generally comprise mixtures of aldehydes, ketones, esters, phenols, acids, and aliphatic, aromatic and other alcohols.

Preservatives also can be added to the compositions of the present invention to prevent bacterial growth. Suitable preservatives approved for use in oral compositions such as methylparaben, propylparaben and sodium benzoate can be added in safe and effective amounts.

Other ingredients can be used in the dentifrice composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

### EXAMPLES

The foregoing may be better understood by reference to the following examples, which are presented for purposes of illustration and are not intended to limit the scope of the invention.

Exemplary silicas of the current invention are prepared as described in the Examples below. Using the analytical methods described below, the silicas of the invention are characterized and the results are providedherein.

Examples 1A-1E. Preparation of Silicates Five dense spherical silicate samples are prepared by spray drying 3.3 MR (molar ratio) and 2.65 MR sodium silicate using the GEA Mobile Minor® spray dryer equipped with a spray nozzle for atomization. The Silicate concentration is fixed at 15 wt. %, The nozzle diameters used is 1.0 mm and 2.0 mm with atomization pressures of 1 bar and 3 bar.

Examples 1F. Preparation of Silicates. Hollow silicate shells are prepared by spray drying 10 wt % 3.3 MR sodium silicate using rotary atomization.

Examples 1A-1I. Preparation of Silica Particles. 300 g of each silicate of Examples 1A-1F are neutralized in 1.8 L of 5.7 wt. % sulfuric acid. Specifically, the solid silicate powder is added slowly over the course of 5 min under stirring at 350 rpm. After 20 min of stirring, the resulting slurry is filtered and washed with 8.0 L of water before subsequent drying at 110° C. for approximately 18 hrs. After neutralization and drying, the silica shells from example 1F are then milled further for 30 sec using a lab scale mechanical mill (Columbia International CIT-FW-200) to form angular/platelets of different particle size, resulting in Example 1G. The neutralization performed in example 1F is repeated to maintain a lower silicate moisture and reaction temperature to provide a lower BET and avoid over acidification. The resulting material is then filtered, dried and mechanically milled following the same procedures as above to provide example 11. These same shells are also further reduced in size using an air jet mill to provide Example 1H.

Example 1J. Silicas of this example is carried out as in Example 1B except that the final pH of the material was signifigantly more alkaline at a pH of 10.18.

The physical properties of the silicas of Examples 1A-1J are measured using the methods below and the results are provided in Table 1 below. Also provided in Table 1 below are properties of Examples 2 and 3 which are conventional silica materials commercially available from Evonik Corporation, which have irregular and non-spherical particle morphology, and Example 4, which are spherical silicas made from a continuous loop reactor process (see e.g., U.S. Pat. Nos. 8,945,517 and 8,609,068).

BET Surface Area. The BET surface areas of silicas of the invention are determined with a Micromeritics TriStar 3020 instrument by the BET nitrogen adsorption method of Brunaur et al., J. Am. Chem. Soc., 60, 309 (1938), which is known in the field of particulate materials, such as silica and silicate materials.

Particle Size. Measurement of the particle size of the silicas of the invention is conducted on HORIBA Laser Scarttering Dry Particle Size Distribution Analyzer LA-930 through the angle of scattered laser light.

Oil Adsorption. Oil absorption values are determined in accordance with the rub-out method described in ASTM D281 using linseed oil (cc oil absorbed per 100 g of the particles). Generally, a higher oil absorption level indicates a higher structure particle, while a lower value typically indicates a lower structure particle.

Pack Density and Pour Density—Pack density and pour density are measured by placing 20.0 g of the sample into a 250 mL graduated cylinder with a flat rubber bottom. The initial volume is recorded and used to calculate the pour density by dividing it into the weight of sample used. The cylinder is then placed onto a tap density machine where it is rotated on a cam at a specific RPM. The cam is designed to raise and drop the cylinder a distance of 5.715 cm once per second, until the sample volume is constant, typically for 15 min. This final volume is recorded and used to calculate the packed density by dividing it into the weight of sample used.

Loss on ignition—LOI are measured by first weighing approximately 2 g sample (to the nearest 0.0001 g) and transfering the sample into a preignited and cooled crucible. The sample must be dried 2 hours at 105° C., cooled to room temperature in a desiccator before weighing. The sample is then heated for 2 hrs in a muffle furnace at a temperature of 1000C. After heating, the sample is removed and cooled in a dessicator. The sample weight is then measured. The % LOI is then calculated by dividing the weight loss by the sample weight before heating.

Acorn surface area—Acorn surface area was measured using a Xigo Acorn Area Surface area Analyzer.

CTAB Surface Area—The CTAB surface areas disclosed herein are determined by absorption of CTAB (cetyltrimethylammonium bromide) on the silica surface, the excess separated by centrifugation and the quantity determined by titration with sodium lauryl sulfate using a surfactant electrode. Specifically, about 0.5 grams of the silica particles are placed in a 250-mL beaker with 100 mL CTAB solution (5.5 g/L), mixed on an electric stir plate for 1 hour, then centrifuged for 30 min at 10,000 RPM. One mL of 10% Triton X-100 is added to 5 mL of the clear supernatant in a 100-mL beaker. The pH is adjusted to 3-3.5 with 0.1 N HCl and the specimen is titrated with 0.01 M sodium lauryl sulfate using a surfactant electrode (Brinkmann SUR1501-DL) to determine the endpoint.

Water Corrected AbC Value. Water absorption values are determined with an Absorptometer “C” torque rheometer from C.W. Brabender Instruments, Inc. Approximately ⅓ of a cup of the silica sample is transferred to the mixing chamber of the Absorptometer and mixed at 150 RPM. Water then is added at a rate of 6 mL/min, and the torque required to mix the powder is recorded. As water is absorbed by the powder, the torque will reach a maximum as the powder transforms from free-flowing to a paste. The total volume of water added when the maximum torque is reached is then standardized to the quantity of water that can be absorbed by 100 g of powder. Since the powder is used on an as received basis (not previously dried), the free moisture value of the powder is used to calculate a “moisture corrected water AbC value” by the following equation.

$$Water Corrected AbC Value = \frac{waterabsorbed ( cc ) + \% moisture}{( 100 ( g ) - \% moisture ) / 100}$$

Mercury intrusion volume—Mercury intruded volume or total pore volume (Hg) is measured by mercury porosimetry using a Micromeritics AutoPore IV 9520 (or, Micromeritics AutoPore V 9620) apparatus. The pore diameters can be calculated by the Washburn equation employing a contact angle Theta (Θ) equal to 1300 and a surface tension gamma equal to 484 dynes/cm. Mercury is forced into the voids of the particles as a function of pressure and the volume of the mercury intruded per gram of sample is calculated at each pressure setting. Total pore volume expressed herein represents the cumulative volume of mercury intruded at pressures from vacuum to 60,000 psi. Increments in volume (cm3/g) at each pressure setting are plotted against the pore radius or diameter corresponding to the pressure setting increments. The peak in the intruded volume versus pore radius or diameter curve corresponds to the mode in the pore size distribution and identifies the most common pore size in the sample. Specifically, sample size is adjusted to achieve a stem volume of 25-90% in a powder penetrometer with a 5 mL bulb and a stem volume of about 1.1 mL. Samples are evacuated to a pressure of 50 μm of Hg and held for 5 minutes. Mercury fills the pores from 4.0 to 60,000 psi with a 10 second equilibrium time at each data collection point). The total pore volume as described above captures the volumes from intraparticle porosity resulting from the pore structure within the individual particles, as well as, the interparticle porosity formed from the interstitial spacing of the packed particles under pressure. To better separate and measure examing the actual intraparticle porosity of the produced amorphous silicas, the pore volume of pores<0.11 μm can be used.

5 wt. % pH. 5% pH is measured by weighing 5.0 g of sample out to the nearest 0.1 g and transferring the weighed sample to a 250 mL beaker. 95 mL of DI water is added and the sample is stirred for 5 min. The pH is then measured with the pH meter while the sample is still stirring.

Einlehner. Einlehner AT-1000 Abrader is used as follows: (1) a Fourdrinier brass wire screen that has been previosly clean and dried is then weighed and exposed to the action of a 10% aqueous silica suspension for a fixed length of time, specifically 100 g of sample in 900 g of deionized water; (2) the amount of abrasion is then determined as milligrams brass lost from the Fourdrinier wire screen per 100,000 revolutions. The result, measured in units of mg loss, can be characterized as the 10% brass Einlehner (BE) abrasion value.

TABLE 1
properties of silicas of Examples 1A-1J, and Examples 2-4

| Example | 1A | 1B | 1C | 1D | 1E | 1F | 1G |
|---|---|---|---|---|---|---|---|
| Nozzle | 1 | 1 | 2 | 2 | 2 | Rotary | Rotary |
| Diameter | | | | | | Atomizer | Atomizer |
| (mm) | | | | | | | |
| Atomization | 1 | 3 | 3 | 1 | 3 | Un-Milled | Mechanically |
| Pressure | | | | | | | Milled |
| (bar) | | | | | | | |
| Silicate Mole | 3.3 | 3.3 | 3.3 | 3.3 | 2.65 | 3.3 | 3.3 |
| Ratio | | | | | | | |
| HgI Total | 0.372 | 0.396 | 0.397 | 0.458 | 0.411 | 2.07 | 0.694 |
| (mL/g) | | | | | | | |
| Intraparticle | 0.029 | 0.001 | 0.016 | 0.046 | 0.008 | 0.315 | 0.070 |
| HgI <0.11 μm | | | | | | | |
| (mL/g) | | | | | | | |
| % Moisture | −0.52 | 0.24 | −0.47 | −0.16 | 0.65 | 2.13 | 2.49 |
| Free Na₂O | 827 | 847 | 561 | 690 | 0.16% | 593 | 850 |
| (ppm) | | | | | | | |
| % Total | 0.15 | 0.19 | 0.11 | 0.24 | 0.18 | 0.11 | 0.14 |
| Na₂O | | | | | | | |
| % Na₂SO₄ | 0.16 | 0.25 | 0.12 | 0.39 | 0.06 | 0.12 | 0.13 |
| % S | 0.037 | 0.056 | 0.026 | 0.089 | 0.013 | 0.027 | 0.028 |
| (Na₂SO₄) | | | | | | | |
| % Quartz | <0.01 | <0.01 | <0.01 | <0.01 | <0.01 | <0.01 | <0.01 |
| BET (m²/g) | 0.6 | 1.7 | 2.0 | 1.0 | 22.3 | 57.3 | 70.4 |
| N₂ | — | 0.0012 | 0.0009 | 0.0002 | 0.0014 | 0.0008 | 0.0070 |
| Adsorption | | | | | | | |
| pore volume | | | | | | | |
| (cm³/g) | | | | | | | |
| N₂ | — | 0.0015 | 0.0010 | 0.0003 | 0.0010 | 0.0010 | 0.0047 |
| Desorption | | | | | | | |
| pore volume | | | | | | | |
| (cm³/g) | | | | | | | |
| % LOI | 7.04 | 8.23 | 7.39 | 7.62 | 6.8 | 8.79 | 8 |
| Acorn (m²/g) | 0.8 | 1.6 | 1.9 | 0.8 | 3.2 | 1.2 | 9 |
| Einlehner mg | 5.23 | 2.43 | 5.9 | 5.92 | 8.67 | 10.58 | 24.78 |
| lost/100k | | | | | | | |
| Rev. | | | | | | | |
| Water AbC | 41 | 35 | 34 | 32 | 35 | 123 | 47 |
| cc/100 g | | | | | | | |
| CTAB | 6 | 12 | 10 | 10 | 1 | −1 | 2 |
| Surface Area | | | | | | | |
| (m2/g) | | | | | | | |
| Pack Density | 69.3 | 67.4 | 69.3 | 62.4 | 69.3 | 27.4 | 59.4 |
| (lb/ft3) | | | | | | | |
| Pour Density | 43.8 | 44.5 | 39 | 39 | 40.2 | 23.1 | 29 |
| (lb/ft3) | | | | | | | |
| Median | 6.12 | 3.73 | 4.45 | 7.78 | 5.95 | 55.28 | 18.54 |
| Particle Size | | | | | | | |
| (μm) | | | | | | | |
| Oil | 17 | 30 | 21 | 18 | 17 | 44 | 31 |
| Absorption | | | | | | | |
| (cc/100 g) | | | | | | | |
| pH 5% | 5.1 | 5.12 | 5.11 | 4.54 | 6.3 | 5.33 | 5.92 |
| DVVA | 40.7 | 41.3 | | 46.8 | | 84.1 | 52.0 |
| VV@15 MPa | | | | | | | |

| Example | 1H | 1I | 1J | 2 | 3 | [symbol] |
|---|---|---|---|---|---|---|
| Nozzle | Rotary | Rotary | 1 | NA | NA | [symbol] |
| Diameter | Atomizer | Atomizer | | | | |
| (mm) | | | | | | |
| Atomization | Air jet | Mechanically | 3 | NA | NA | [symbol] |
| Pressure | Milled | Milled | | | | |
| (bar) | | | | | | |
| Silicate Mole | 3.3 | 3.3 | 3.3 | NA | NA | [symbol] |
| Ratio | | | | | | |
| HgI Total | 0.754 | 0.738 | 0.601 | 0.92 | 1.48 | 0.75 |
| (mL/g) | | | | | | |
| Intraparticle | 0.011 | 0.022 | 0.047 | 0.335 | 0.649 | 0.193 |
| HgI <0.11 μm | | | | | | |
| (mL/g) | | | | | | |
| % Moisture | | | | 5.0 | 6.8 | 4.3 |
| Free Na₂O | 0.49% | 0.49% | 1.67% | | | |
| (ppm) | | | | | | |
| % Total | 0.60 | 0.58 | 1.96 | 1.25 | 0.98 | 0.96 |
| Na₂O | | | | | | |
| % Na₂SO₄ | 0.25 | 0.22 | 0.65 | 2.08 | 1.14 | 0.82 |
| % S | | | | | | |
| (Na₂SO₄) | | | | | | |
| % Quartz | <0.01 | <0.01 | | <0.01 | <0.01 | <0.01 |
| BET (m²/g) | 9.2 | 5.1 | 1.3 | 56.4 | 77.4 | 89 |
| N₂ | 0.0177 | 0.0131 | 0.0035 | 0.1954 | 0.2876 | 0.1938 |
| Adsorption | | | | | | |
| pore volume | | | | | | |
| (cm³/g) | | | | | | |
| N₂ | 0.0183 | 0.0129 | 0.0035 | 0.2055 | 0.2927 | 0.2095 |
| Desorption | | | | | | |
| pore volume | | | | | | |
| (cm³/g) | | | | | | |
| % LOI | | | | 4.1 | | 3.8 |
| Acorn (m²/g) | | | | 7.30 | 18.4 | 11.1 |
| Einlehner mg | 17.69 | | | 15.2 | 3.7 | 1.4 |
| lost/100k | | | | | | |
| Rev. | | | | | | |
| Water AbC | | | | 70 | 108 | 75 |
| cc/100 g | | | | | | |
| CTAB | 9 | 6 | 4 | 63 | 66 | 56 |
| Surface Area | | | | | | |
| (m2/g) | | | | | | |
| Pack Density | 41.6 | 54.3 | 56.8 | 45.0 | 28.0 | 46.8 |
| (lb/ft3) | | | | | | |
| Pour Density | 23.1 | 34.7 | 33.7 | 26.0 | 18.0 | 30.2 |
| (lb/ft3) | | | | | | |
| Median | 3.5 | 13.3 | 4.0 | 9.7 | 9.14 | 3.5 |
| Particle Size | | | | | | |
| (μm) | | | | | | |
| Oil | 47 | | | 53 | 81 | 66 |
| Absorption | | | | | | |
| (cc/100 g) | | | | | | |
| pH 5% | 9.11 | 9.39 | 10.18 | 7.43 | 7.33 | 7.2 |
| DVVA | 60 | | | 53.0 | 78.6 | 62.9 |
| VV@15 MPa | | | | | | |

[symbol]

indicates data missing or illegible when filed

As can be seen in Table 1, the amorphous silica particles produced by the process of the invention utilizing the spray nozzle have a very low BET surface area, low medican particle size, low oil absorption capacity as well as high pack density (great than 60 lb/ft³) relative to silica particles made from the process using the rotary atomizer. It can also be seen that the amorphous silica particles produced via the process of the invention utilizing the rotary atomizer can also have a very low BET surface area, low oil absorption capacity and relatively high pack densities (greater than 40 lb/ft³ if milled to an appropriately small size) as seen in Example 1H and 11. This indicates that similar to the dense spheres, the resulting particles from this process can be non-porous.

Examples 5A-C. The following examples show the impact of neutralization conditions on the final properties of the silica materials.

Hollow silicate shells are prepared from 35 wt. % 2.5 MR sodium silicate using rotary atomization. 100 g of silicate is then neutralized in 600 mL of 5.7 wt % sulfuric acid under different temperatures. Specifically, the solid silicate powder is added slowly over the course of 5 min under stirring at 350 rpm at acid temperatures of room temperature, 30° C., and 40° C. After 20 min of stirring, the resulting slurry is filtered and washed with 3.0 L of water before subsequent drying at 110° C. for approximately 18 hrs. After drying, the materials are only lightly milled in a Cuisinart mixer to break up large agglomerates. The results of Examples 5A-C are shown in Table 2 below:

TABLE 2

| Example | 5A | 5B | 5C |
|---|---|---|---|
| Nozzle Diameter (mm) | Rotary | Rotary | Rotary |
| | Atomizer | Atomizer | Atomizer |
| Silicate Mole Ratio | 2.5 | 2.5 | 2.5 |
| Neutralization Temperature | 40° C. | 30° C. | Room |
| | | | temperature |
| Free Na2O (ppm) | 94 | 0 | 0 |
| % Total Na2O | 0.23 | 0.3 | 0.22 |
| % Na2SO4 | 0.51 | 0.72 | 0.5 |
| % Moisture | 0.02 | 0 | 0 |
| AbC | 198 | 222 | 226 |
| BET (m2/g) | 315 | 80 | 22 |
| CTAB Surface Area (m2/g) | 53 | 6 | 0 |
| Median Particle Size (μm) | 65.6 | 64.6 | 64.2 |
| Oil Absorption (cc/100 g) | 91 | 75 | 75 |
| pH 5% | 3.23 | 3.05 | 3.51 |

The results in Table 2 shows that BET surface area increased at elevated temperature. Not to be bound by any hypothesis, it is believed that the increase in surface area results from increased alkali metal silicate solubility and this can be mitigated by maintaining a lower reaction temperature. Similar increases in reaction temperature can be seen from the exothermic reaction of the alkali metal silicate and room temperature acid. Therefore, it was found that maintaining raw material properties and neutralization conditions that provide an overall lower average reaction temperature and minimize this solubility at a given reaction temperature are needed to provide the desired lower BET surface areas.

Stannous Compatibility Test. Using the methods described below, various silicas of the present invention previuosly shown in Table 1, are tested for stannous and fluoride compatibility. The results of which are shown in Table 3. The same stannous and fluoride compatbility tests are also carried out for Examples 1A′ through 1D′ which are prepared by adjusting the pH (5 wt. %) of the silicas of Examples 1A through 1D to 7.1 with sodium bicarbonate.

Stannous compatibility (%). The Stannous compatibility of the above samples are determined as follows. A stock solution containing 431.11 g of 70% sorbitol, 63.62 g of de-oxygenated deionized water, 2.27 g of stannous chloride dihydrate, and 3 g of sodium gluconcate is prepared. 34 g of the stock solution is added to a 50 mL centrifuge tube containing 6 g of the silica sample to be tested. The centrifuge tube is placed on a rotating wheel at 5 RPM and is aged for 1 week at 40° C. After aging, the centrifuge tube is centrifuged at 12,000 RPM for 10 minutes, and the stannous concentration in the supernatant is determined by ICP-OES (inductively coupled plasma optical emission spectrometer). The stannous compatibility is determined by expressing the stannous concentration of the sample as a percentage of the stannous concentration of a solution prepared by the same procedure, but with no silica added.

Fluoride Compatibility (%). The Fluoride compatibility of the above samples are determined as follows. A fluoride stock solution containing 2.21 g of sodium fluoride, 4.15 g of citric acid, 23.05 g of sodium citrate dihydrate, and diluted to total volume of 1000 mL using deionized water is prepared. A 40 wt % silica test slurry is created using the material of interest and the fluoride stock solution. 11.25 g of the stock solution is added to a 50 mL centrifuge tube containing 7.5 g of the silica sample to be tested. The centrifuge tube is placed on a rotating wheel at 5 RPM (or equivalent means of agitation) and is aged for 1 hour at 60° C. After aging, the centrifuge tube is centrifuged at 12,000 RPM for 10 minutes (or until the supernatant is clear). The Fluoride concentration in the supernatant is determined by first taking an aliquot, transferring it to a plastic vial containing a magnetic stirbar and adding an equal volume of TISAB II buffer. The concentration is then measured using a pre-calibrated fluoride specific ion electrode (orion model #96-09BN or equivalent). The Fluoride compatibility is determined by expressing the Fluoride concentration of the sample as a percentage of the Fluoride concentration of a stock solution.

TABLE 3

| | Sn | F | | |
|---|---|---|---|---|
| | Compatibility | Compatibility | BET | pH |
| Example | (%) | (%) | (m²/g) | (5 wt %) |
| 2 | <40 | 74.0 | 56.4 | 7.43 |
| 3 | <40 | 74.9 | 77.4 | 7.33 |
| 4 | <40 | 73.7 | 89 | 7.2 |
| 1A | 96 | 74.1 | 0.6 | 5.1 |
| 1B | 94 | 84.4 | 1.7 | 5.12 |
| 1C | 95 | 71.8 | 2.0 | 5.11 |
| 1D | 97 | 70.7 | 1.0 | 4.54 |
| 1F | 82.3 | — | 57.3 | 5.33 |
| 1G | 66.9 | 67.0 | 70.4 | 5.92 |
| 1H | — | — | 9.1 | 9.11 |
| 1I | — | 92.0 | 5.1 | 9.39 |
| 1J | — | 100 | 1.3 | 10.18 |
| 1A′ | 87 | — | 0.6 | 9.96 |
| 1B′ | 92 | — | 1.7 | 10.29 |
| 1C′ | 86 | — | 2.0 | 10.30 |
| 1D′ | 88 | 94.5 | 1.0 | 10.26 |
| 1F′ | 96.1 | — | 57.3 | — |
| 1G′ | 91.3 | 75.3 | 70.4 | 8.1 |

The results in Table 3 show that the silicas of the present invention have improved fluoride and stannous compatibility compared to commercially available high cleaning silica of Examples 2, 3 and 4. Unexpectedly from the prior art (U.S. Pat. Nos. 3,939,262 and 4,312,845), which described materials with higher BET surface areas, it was found that at a given pH range (for example>7), having a lower BET surface area is important in maintaining the maximum Fluoride compatibility. This can be seen when comparing the Fluoride compatibilities of Example 1 G′ to those of Examples 1I, 1J and 1D′.

Silicas of the invention for oral care application. Examples DC1-DC14. The dentrifice compositions of Examples 001-0014 are prepared by using a IKA overhead stirrer to first mix a gum dispersion containing polyethylene glycol, glycerin, and sorbitol with an aqueus solution containing fluoride, sweetener, preservative, a whitening agent, and color. Various amounts of silicas of Examples 1B, 1D, 1B′, 1H, 1I and 1 J are then added to this pre-mix as shown in Table 4 and the resulting mixture is placed in a Ross Dual Planetary Mixture and mixed for 15 min to 25 min under strong vacuum. Flavor and surfactant are added and the material is mixed for another 5 min under low vacuum. The specific mass of each ingredient is reported in Table 4.

TABLE 4

| Dentifrice | | | | | | | | |
|---|---|---|---|---|---|---|---|---|
| Composition | DC 1 | DC 2 | DC 3 | DC 4 | DC 5 | DC 6 | DC 7 | DC 8 |
| Glycerin, 99.7% (g) | 11.000 | 11.000 | 13.160 | 13.160 | 11.000 | 13.160 | 14.500 | 11.000 |
| Sorbitol, 70.0% (g) | 40.007 | 40.007 | 42.177 | 42.177 | 40.007 | 42.177 | 43.507 | 40.007 |
| Deionized Water (g) | 20.000 | 20.000 | 22.170 | 22.170 | 20.000 | 22.170 | 23.500 | 20.000 |
| PEG-12 (g) | 3.000 | 3.000 | 3.000 | 3.000 | 3.000 | 3.000 | 3.000 | 3.000 |
| Cekol 2000 | 1.200 | 1.200 | 1.200 | 1.200 | 1.200 | 1.200 | 1.200 | 1.200 |
| AA6458332 (g) | | | | | | | | |
| Tetrasodium | 0.500 | 0.500 | 0.500 | 0.500 | 0.500 | 0.500 | 0.500 | 0.500 |
| Pyrophosphate (g) | | | | | | | | |
| Sodium Saccharin (g) | 0.200 | 0.200 | 0.200 | 0.200 | 0.200 | 0.200 | 0.200 | 0.200 |
| Sodium Fluoride (g) | 0.243 | 0.243 | 0.243 | 0.243 | 0.243 | 0.243 | 0.243 | 0.243 |
| Zeodent 165 | 1.500 | 1.500 | 5.000 | 5.000 | 1.500 | 5.000 | 10.000 | 1.500 |
| (Thickener) (g) | | | | | | | | |
| Example 2 (Control) | 20.000 | | | | | | | |
| (g) | | | | | | | | |
| Example 3 (Control) | | 20.000 | | | | | | |
| (g) | | | | | | | | |
| Example 1B (g) | | | 10.000 | | | | | |
| Example 1D (g) | | | | 10.000 | | | | |
| Example 1B′ (g) | | | | | 20.000 | 10.000 | 1.000 | |
| Example 1H | | | | | | | | 20.000 |
| Example 1I | | | | | | | | |
| Example 1J | | | | | | | | |
| Example 4 (Control) | | | | | | | | |
| (g) | | | | | | | | |
| TiO2 (g) | 0.500 | 0.500 | 0.500 | 0.500 | 0.500 | 0.500 | 0.500 | 0.500 |
| Sodium Lauryl | 1.200 | 1.200 | 1.200 | 1.200 | 1.200 | 1.200 | 1.200 | 1.200 |
| Sulfate (g) | | | | | | | | |
| Flavor (g) | 0.650 | 0.650 | 0.650 | 0.650 | 0.650 | 0.650 | 0.650 | 0.650 |
| Total (g) | 100.000 | 100.000 | 100.000 | 100.000 | 100.000 | 100.000 | 100.000 | 100.000 |

| Dentifrice | | | | | | | |
|---|---|---|---|---|---|---|---|
| Composition | DC 9 | DC 10 | DC 11 | DC 12 | DC 13 | DC 14 | DC 15 |
| Glycerin, 99.7% (g) | 13.160 | 11.000 | 13.160 | 11.000 | 13.160 | 14.500 | 11.000 |
| Sorbitol, 70.0% (g) | 42.177 | 40.007 | 42.177 | 40.007 | 42.177 | 43.507 | 40.007 |
| Deionized Water (g) | 22.170 | 20.000 | 22.170 | 20.000 | 22.170 | 23.500 | 20.000 |
| PEG-12 (g) | 3.000 | 3.000 | 3.000 | 3.000 | 3.000 | 3.000 | 3.000 |
| Cekol 2000 | 1.200 | 1.200 | 1.200 | 1.200 | 1.200 | 1.200 | 1.200 |
| AA6458332 (g) | | | | | | | |
| Tetrasodium | 0.500 | 0.500 | 0.500 | 0.500 | 0.500 | 0.500 | 0.500 |
| Pyrophosphate (g) | | | | | | | |
| Sodium Saccharin (g) | 0.200 | 0.200 | 0.200 | 0.200 | 0.200 | 0.200 | 0.200 |
| Sodium Fluoride (g) | 0.243 | 0.243 | 0.243 | 0.243 | 0.243 | 0.243 | 0.243 |
| Zeodent 165 | 5.000 | 1.500 | 5.000 | 1.5 | 5 | 10 | 1.500 |
| (Thickener) (g) | | | | | | | |
| Example 2 (Control) | | | | | | | |
| (g) | | | | | | | |
| Example 3 (Control) | | | | | | | |
| (g) | | | | | | | |
| Example 1B (g) | | | | | | | |
| Example 1D (g) | | | | | | | |
| Example 1B′ (g) | | | | | | | |
| Example 1H | 10.000 | | | | | | |
| Example 1I | | 20.000 | 10.000 | | | | |
| Example 1J | | | | 20.000 | 10.000 | 1.000 | |
| Example 4 (Control) | | | | | | | 20.000 |
| (g) | | | | | | | |
| TiO2 (g) | 0.500 | 0.500 | 0.500 | | | | 0.500 |
| Sodium Lauryl | 1.200 | 1.200 | 1.200 | | | | 1.200 |
| Sulfate (g) | | | | | | | |
| Flavor (g) | 0.650 | 0.650 | 0.650 | | | | 0.650 |
| Total (g) | 100.000 | 100.000 | 100.000 | 100.000 | 100.000 | 100.000 | 100.000 |

Relative Dentin Abrasion (RDA). The RDA values of dentifrice compositions of Examples DC1-DC14 containing the silicas of the current invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, the contents of which are incorporated herein by reference in their entirety.

Pellicle Cleaning Ratio (“PCR”). The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain with Dentifrice” G. K. Stookey, et al., J. Dental Res., 61, 12-36-9, 1982. Both PCR and RDA results vary depending upon the nature and concentration of the components of the dentifrice composition. PCR and RDA values are unit-less. Both PCR and RDA values are obtaing and are reported below in Table 5.

TABLE 5

| Dentifrice | Test | | Loading | | |
|---|---|---|---|---|---|
| Composition | Set | Silica Abrasive | (%) | PCR | RDA |
| DC 1 | 1 | 2 (Control) | 20 | 100 | 194 |
| DC 2 | 1 | 3 (Control) | 20 | 73 | 100 |
| DC 3 | 1 | 1B | 10 | 95 | 143 |
| DC 4 | 1 | 1D | 10 | 93 | 125 |
| DC 5 | 1 | 1B′ | 20 | 106 | 160 |
| DC 6 | 1 | 1B′ | 10 | 96 | 134 |
| DC 7 | 1 | 1B′ | 1 | 75 | 57 |
| DC 1 | 2 | Sample 2 (Control) | 20 | 112 | 208 |
| DC 2 | 2 | Sample 3 (Control) | 20 | 77 | 111 |
| DC8 | 2 | 1H | 20 | 114 | 147 |
| DC9 | 2 | 1H | 10 | 106 | 143 |
| DC10 | 2 | 1I | 20 | 111 | 198 |
| DC11 | 2 | 1I | 10 | 113 | 168 |
| DC12 | 2 | 1J | 20 | 114 | 147 |
| DC13 | 2 | 1J | 10 | 89 | 53 |
| DC14 | 2 | 1J | 1 | 98 | 62 |
| DC 15 | 3 | 4 (Control) | 20 | 118 | 270 |

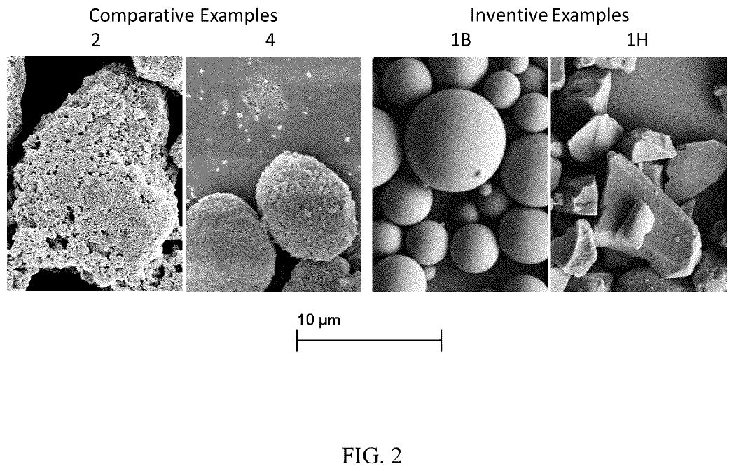

Table 5 shows that silicas of Examples DC 3-6 have improved PCR relative to control silicas of Example 3 as well as comparable PCR and improved RDA relative to control silca of Example 2. The same holds true for the angular/platelet materials Examples DC8-11, but it was observed that reducing the particle size is critical to minimizing the RDA when utilizing this morphology. The reduced RDA values achieved relative to other precipitated silica materials with similar particle densities and sizes was unexpected. Not to be bound by a particular hypothesis, it is believed that the lack of any porosity leads to a increased particle surface uniformity and smoothness, which can De seen in SEM imaging (shown in FIG. 2), which alters their interaction with and ultimately their abrasion of relavent oral surfaces.

Relative Enamel Abrasivity (“REA”). The silica particles of the invention possess a lower degree of polymerization and particle hardness as a result of not being heat treated or fused, which helps them to maintain a lower abrasivity toward other hard surfaces such as metals and even enamel. REA test of commercially available fused silica particles of similar size and shape as Example 1B and 1H along with traditional high cleaning precipitated silica are tested for their REA values. As can be seen, the higher hardness resulting from the heat treatment process of the fused silica particles results in undesirably high brass einlehner abrasion values and correspondingly high REA values compared to the traditional high cleaning precipitated silica. On the other hand, the lower degree of polymerization in the non-heat treated silica particles of Examples 1B and 1H help them to maintain a lower brass einlehner abrasion, which are expected to have a lower and more desirable REA value.

TABLE 6

| | Toothpaste | | Median | | |
|---|---|---|---|---|---|
| | Loading | | Particle | | |
| Silica Abrasive | Level (%) | Morphology | Size (μm) | REA | Einlehner |
| Example 2 (Control) | 20 | Irregular/angular | 9.5 | 5.44 | 15.24 |
| 1B | | Spherical | 3.7 | | 2.41 |
| 1H | | Angular | 3.5 | | 17.69 |
| PEMCO 325/F10 | 15 | Angular | 3.51 | 19.83 | 175.26 |
| Fused Silica | | | | | |
| PEMCO 325/F20 | 15 | Angular | 4.51 | 18.85 | 380.35 |
| Fused Silica | | | | | |
| CE Minerals Teco- | 15 | Angular | 4.55 | 24.03 | 208.39 |
| sil 44C | | | | | |
| CE Minerals Teco- | 15 | Angular | 3.72 | 24.17 | 161.88 |
| sil 44CSS | | | | | |

1: An oral composition comprising;
amorphous, non-porous silica particles that are not produced via polycondensation of a tetraalkoxysilane or a precipitation process or heat treated to over 200° C.,
wherein the relative dentine abrasive (RA) value is less than 200, and
wherein the silica particles have a total mercury intrusion (HgI) pore volume of less than 0.8 ml/g or an intraparticle HgI pore volume of pores less than 0.11 micron in diameter is less than 0.10 cc/a.

2: The oral composition of claim 1, wherein the silica particles are spherical.

3: The oral composition of claim 1, wherein said silica particles have one or more of the following properties:
(a) a BET surface area of the silica particles is less than 25 m²/g;
(b) a median particle size of the silica particles is between 1 and 25 μm;
(c) an oil absorption capacity of the silica particles is below or equal to 40 cc/100 g;
(d) a pack density of the silica particles is greater than 60 lb/ft³;
(e) a pour density of the such silica particles is less than 50 lb/ft³;
(f) a loss on ignition (LOI) of the silica particles is between 3 and 10 wt. %;
(g) an Acorn surface area of the silica particles is less than 10 m²/g;
(h) a CTAB surface area of the silica particles is less than 15 m²/g;
(i) the total mercury intrusion pore volume of the silica particles is less than 0.7 cc/g;
(j) the intraparticle mercury intrusion volume of pores less than 0.11 microns in diameter of the silica particles is less than 0.10 cc/g;
(k) a 5% pH is between 4 and 11;
(l) a moisture corrected water AbC value of the silica particles is less than cc/100 g;
(m) an Einlehner abrasion value of the silica particles is less than or equal to 10 mg loss per 100,000 revolutions;
(n) a BJH nitrogen adsorption pore volume of the silica particles is less than 0.002 cc/g; and/or
(o) any combination of (a)-(n) thereof.

4: The oral composition of claim 1, wherein the silica particles are non-spherical and have one or more of the following properties:
(a) a BET surface area is less than 25 m²/g;
(b) a median particle size is between 1 and 25 μm;
(c) an oil absorption capacity is less than 50 cc/10 g;
(d) a pack density is greater than 35 lb/ft³;
(e) a pour density of the silica particles is less than 40 lb/ft³;
(f) a loss on ignition (LOI) is between 3 and 10 wt. %;
(g) an Acorn surface area is less than 10 m²/g;
(h) a CTAB surface area of the silica particles is less than 15 m²/g;
(i) the total mercury intrusion pore volume is less than or equal to 0.8 cc/g;
(j) the intraparticle mercury intrusion pore volume of pores less than 0.11 microns in diameter of the silica particles is less than 0.10 cc/g;
(k) a 5% pH is between 4 and 11;
(l) a moisture corrected water AbC value is less than 90 cc/100 g;
(m) an Einlehner abrasion value is less than or equal to 30 mg loss per 100,000 revolutions;
(n) a BJH nitrogen adsorption pore volume of the silica particles is less than 0.02 cc/g; and/or
(o) any combination of (a)-(n) thereof.

5: The oral composition of claim 1, wherein a Scanning Electron Microscopy (SEM) and/or Transmission Electron Microscopy (TEM) of the silica particles shows no evidence of an internal structure or porosity.

6: The oral composition of claim 1, wherein the composition is an oral care composition comprising one or more orally acceptable carriers.

7: The oral composition of claim 1, wherein a loading level of silica is between 1 and 20 wt. %, based on a total weight of the composition.

8: The oral composition of claim 1, wherein a Pellicle Cleaning Ratio (PCR) is greater than 70.

9: The oral composition of claim 1, wherein the relative dentine abrasive (RDA) value is less than or equal to 180.

10: The oral composition of claim 1, wherein said composition has a fluoride compatibility of greater than or equal to 60%.

11: The oral composition of claim 1, wherein said composition has a stannous ion compatibility of greater than or equal to 50%.

12: The oral composition of claim 1, wherein a Relative Enamel abrasion is less than or equal to 15.

13: The oral composition of claim 1, wherein the silica particles are made from a process comprising:
(i) spray drying an alkali metal silicate solution to form silicate powder; and
(ii) neutralizing the silicate powder of (i) with an acid.

14: The oral composition of claim 13, wherein the spray drying (i) comprises atomization of a sodium silicate using a spray nozzle or a pneumatic nozzle.

15: The oral composition of claim 13, wherein the spray drying (i) comprises atomization using a rotary atomizer.

16: The oral composition of claim 13, wherein a neutralization temperature of the alkali metal silicate is less than or equal to 30° C.

17: The oral composition of claim 13 any of claims 13, wherein:
a. a molar ratio of silica: alkali metal oxide is between 2.5 and 3.5;
b. the alkali metal is selected from the group consisting of sodium, potassium, lithium, and any mixture thereof;
c. a silicate concentration is between 10 and 15 wt. %;
d. the acid is a mineral acid; and/or
e. the acid is at a concentration of less than 10 wt.

18: The oral composition of claim 13, wherein milling is required.

19: The oral composition of claim 13, wherein no milling is required.

20: The oral composition of claim 13, wherein the acid is adjusted to provide a final pH of dried silica of greater than or equal to 7.
